(19) United States
(12) Patent Application Publication
(10) Pub. No.: US 2021/0197400 A1
(43) Pub. Date: Jul. 1, 2021

(54) INSTRUMENT DRIVE MECHANISM FOR ROBOTICS

(71) Applicant: KINOVA INC., Boisbriand (CA)

(72) Inventors: Sebastien MESSIER, Boisbriand (CA); Philippe FAUTEUX, Boisbriand (CA); Sebastien BOISVERT, BOISBRIAND (CA); Eric LAVIGNE, BOISBRIAND (CA); Jean-Luc BOUCHARD, BOISBRIAND (CA)

(21) Appl. No.: 16/756,878

(22) PCT Filed: Oct. 19, 2018
(86) PCT No.: PCT/CA2018/051320

§ 371 Date: Apr. 17, 2020

Related U.S. Application Data
(60) Provisional application No. 62/574,512, filed on Oct. 19, 2017.

(51) Int. Cl. B25J 15/02; F16H 37/04; G01D 5/14; G01D 5/26; G01J 5/00; H02K 7/116; H02K 11/00; H02K 11/22

(52) U.S. Cl. CPC B25J 15/02; F16H 37/04; G01D 5/14; G01D 5/26; A61M 25/0116; H02K 7/116; H02K 11/0094; H02K 11/22; G01J 5/0022

(57) ABSTRACT

An instrument drive mechanism comprises an outer shell having an open-ended receptacle. An internal gear is secured inside the open-ended receptacle and immovable relative to the outer shell. An interface cover is rotatably mounted to the open-ended receptacle, the interface cover configured to be connected to an instrument, the interface cover rotatably supporting at least one cover shaft with an output adapted to be rotatingly coupled to the instrument. A drive system is rotatably mounted to the open-ended receptacle and connected to the interface cover to rotate with the interface cover, the drive system having at least two motor units, a coupling assembly between each of the at least one cover shaft and a corresponding one of the motor units for releasably coupling a motor unit shaft to the cover shaft, for the at least one said motor unit coupled to each said at least one cover shaft to transmit a degree of actuation thereto, and one said motor unit having a gear coupled to internal gear to drive a rotation of the interface cover and drive system relative to the outer shell.

### CROSS-REFERENCE TO RELATED APPLICATION

The present application claims the priority of U.S. Patent Application No. 62/574,512, filed on Oct. 19, 2017, and incorporated herein by reference.

### TECHNICAL FIELD

The present application relates to robot arms and to an instrument drive mechanism that interfaces and drives an end-effector instrument for instance at an end of a robot arm.

### BACKGROUND OF THE ART

Robotic arms are increasingly used in a number of different applications, from manufacturing, to servicing, and assistive robotics, among numerous possibilities. Serial robot arms are convenient in that they cover wide working volumes. The instruments at the effector end of robot arms may be self-operated, or may be connected to a drive mechanism that may control the instruments in different ways, such as adjusting their position and/or orientation, drive the operation of the instrument, etc. There are consequently drive mechanisms that may provide several degrees of actuation, by incorporating numerous motors and associated components. The challenge remains to package the numerous motors and associated components in a compact manner, for the drive mechanism to have a reduced volume to operate in limited spaces, while preserving their capability of performing multiple controlling actions according to precision standards.

### SUMMARY

It is an aim of the present disclosure to provide an instrument drive mechanism that addresses issues related to the prior art.

Therefore, in accordance with a first embodiment of the present disclosure, there is provided an instrument drive mechanism comprising: an outer shell having an open-ended receptacle; an internal gear secured inside the open-ended receptacle and immovable relative to the outer shell; an interface cover rotatably mounted to the open-ended receptacle, the interface cover configured to be connected to an instrument, the interface cover rotatably supporting at least one cover shaft with an output adapted to be rotatingly coupled to the instrument; a drive system rotatably mounted to the open-ended receptacle and connected to the interface cover to rotate with the interface cover, the drive system having at least two motor units, a coupling assembly between each of the at least one cover shaft and a corresponding one of the motor units for releasably coupling a motor unit shaft to the cover shaft, for the at least one said motor unit coupled to each said at least one cover shaft to transmit a degree of actuation thereto, and one said motor unit having a gear coupled to internal gear to drive a rotation of the interface cover and drive system relative to the outer shell.

Further in accordance with the first embodiment, as an example, the coupling assembly includes at least a coupler connected to the cover shaft, and a coupler receiving a drive from the motor unit.

Still further in accordance with the first embodiment, as an example, each of said coupling assembly is an Oldham coupling.

Still further in accordance with the first embodiment, as an example, each motor unit has a motor and a reduction gear box (RGB) connected to the motor, a RGB shaft being coupled to the cover shaft by the coupling assembly.

Still further in accordance with the first embodiment, as an example, each said cover shaft is connected to the interface cover by at least one bearing.

Still further in accordance with the first embodiment, as an example, at least two of the cover shaft are for instance provided, each with one said output, with one said coupling assembly between each of the two cover shafts and a corresponding one of the motor units.

Still further in accordance with the first embodiment, as an example, the interface cover, the at least one shaft and a coupler of coupling assembly form a cartridge removable as a group from the outer shell and from engagement with the drive system.

Still further in accordance with the first embodiment, as an example, at least one bearing is between an inner surface of the outer shell and a periphery of the interface cover.

Still further in accordance with the first embodiment, as an example, a central shaft extends into the outer shell and rotatably supported by the outer shell, the interface cover and the drive system coupled to the central shaft to rotate concurrently with the central shaft.

Still further in accordance with the first embodiment, as an example, a sensor unit has a sensor portion mounted onto a printed circuit board (PCB) connected to the drive system and/or to the shaft to monitor a rotation of the shaft and/or of the drive system relative to the outer shell to determine an angular position of the interface cover relative to the outer shell.

Still further in accordance with the first embodiment, as an example, the sensor portion of the PCB is a magnetic sensor.

Still further in accordance with the first embodiment, as an example, a magnetic ring is secured to the outer shell and surrounding the central shaft adjacent to the magnetic sensor.

Still further in accordance with the first embodiment, as an example, the magnetic sensor and the magnetic ring lie in a common radial plane of the central shaft.

Still further in accordance with the first embodiment, as an example, the magnetic sensor is radially outward of the magnetic sensor.

Still further in accordance with the first embodiment, as an example, the interface cover has a central bore, the central bore of the interface cover forming a continuous passage with an inner cavity of the central shaft.

Still further in accordance with the first embodiment, as an example, at least one printed circuit board is connected to the drive system, the printed circuit board supporting a temperature sensor for each said motor unit, and an optical encoder for each said motor unit to determine an angular position of each said output on the interface cover.

Still further in accordance with the first embodiment, as an example, the temperature sensor is an infrared temperature sensor.

Still further in accordance with the first embodiment, as an example, the infrared temperature sensor is aligned with a shaft of its corresponding motor unit.

Still further in accordance with the first embodiment, as an example, a pad rotates with a shaft of the motor unit, the pad paired with the optical encoder.

Still further in accordance with the first embodiment, as an example, the optical encoder is located offset relative to a center of a shaft of the motor unit.

In accordance with a second embodiment of the present disclosure, there is provided an instrument drive mechanism comprising: an outer shell having an open-ended receptacle; an internal gear secured inside the open-ended receptacle and immovable relative to the outer shell; a central shaft extending into the outer shell and rotatably supported by the outer shell; an interface cover coupled to the central shaft and rotatably mounted to the open-ended receptacle, the interface cover configured to be connected to an instrument, the interface cover rotatably supporting at least one cover shaft with an output adapted to be rotatingly coupled to the instrument; a drive system rotatably mounted to the open-ended receptacle and connected to the interface cover and to the central shaft to rotate with the interface cover, the drive system having at least two motor units, at least one said motor unit coupled to said at least one cover shaft to transmit a degree of actuation thereto, and one said motor unit having a gear coupled to internal gear to drive a rotation of the interface cover and drive system relative to the outer shell; and a sensor unit having a sensor portion mounted onto a printed circuit board (PCB) connected to the drive system and/or to the shaft to monitor a rotation of the shaft and/or of the drive system relative to the outer shell to determine an angular position of the interface cover relative to the outer shell. Further in accordance with the second embodiment, as an example, the sensor portion of the PCB is a magnetic sensor.

Still further in accordance with the second embodiment, as an example, a magnetic ring may be secured to the outer shell and surrounding the central shaft adjacent to the magnetic sensor.

Still further in accordance with the second embodiment, as an example, the magnetic sensor and the magnetic ring lie in a common radial plane of the central shaft.

Still further in accordance with the second embodiment, as an example, the magnetic sensor is radially outward of the magnetic sensor.

Still further in accordance with the second embodiment, as an example, the interface cover has a central bore, the central bore of the interface cover forming a continuous passage with an inner cavity of the central shaft.

In accordance with a third embodiment of the present disclosure, there is provided an instrument drive mechanism comprising: an outer shell having an open-ended receptacle; an internal gear secured inside the open-ended receptacle and immovable relative to the outer shell; a central shaft extending into the outer shell and rotatably supported by the outer shell; an interface cover coupled to the central shaft and rotatably mounted to the open-ended receptacle, the interface cover configured to be connected to an instrument, the interface cover rotatably supporting at least one cover shaft with an output adapted to be rotatingly coupled to the instrument; a drive system rotatably mounted to the open-ended receptacle and connected to the interface cover and to the central shaft to rotate with the interface cover, the drive system having at least two motor units, at least one said motor unit coupled to said at least one cover shaft to transmit a degree of actuation thereto, and one said motor unit having a gear coupled to internal gear to drive a rotation of the interface cover and drive system relative to the outer shell; and at least one printed circuit board connected to the drive system, the printed circuit board supporting a temperature sensor for each said motor unit, and an optical encoder for each said motor unit to determine an angular position of each said output on the interface cover.

Further in accordance with the third embodiment, as an example, the temperature sensor is an infrared temperature sensor.

Still further in accordance with the third embodiment, as an example, the infrared temperature sensor is aligned with a shaft of its corresponding motor unit.

Still further in accordance with the third embodiment, as an example, a pad may rotate with a shaft of the motor unit, the pad paired with the optical encoder.

Still further in accordance with the third embodiment, as an example, the optical encoder is located offset relative to a center of a shaft of the motor unit.

### DESCRIPTION OF THE DRAWINGS

FIG. 1 is a perspective view of an instrument drive mechanism for robotics in accordance with the present disclosure;

FIG. 2A is a longitudinal sectional view of the instrument drive mechanism of FIG. 1;

FIG. 2B is zoom view of a part of the longitudinal sectional view of FIG. 2A of the instrument drive mechanism;

FIG. 3 is a longitudinal sectional view of the instrument drive mechanism of FIG. 1, with a interface cover removed;

FIG. 4 is an assembly view of group components of the instrument drive mechanism of FIG. 1;

FIG. 5 is an assembly view of driving components of the instrument drive mechanism of FIG. 1;

FIG. 6 is a side view of the components of the instrument drive mechanism of FIG. 5;

FIG. 7 is a perspective view of the components of the instrument drive mechanism of FIG. 6;

FIG. 8 is a perspective view of an exemplary articulated robotic arm used with the instrument drive mechanism of FIG. 1;

FIG. 9 is a perspective view of an arrangement of sensor boards of the instrument drive mechanism of FIG. 1; and

FIG. 10 is a schematic view showing an end of a motorized joint unit of the instrument drive mechanism of FIG. 1.

### DETAILED DESCRIPTION

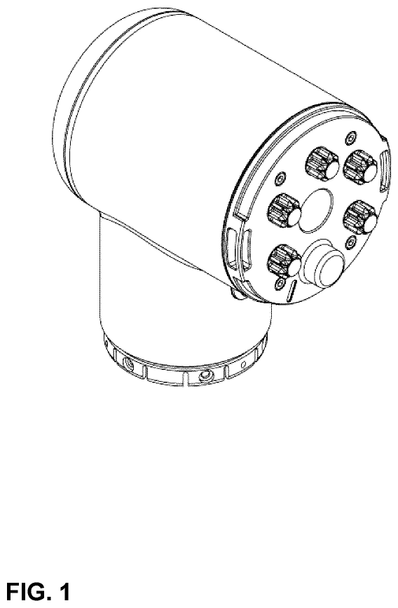

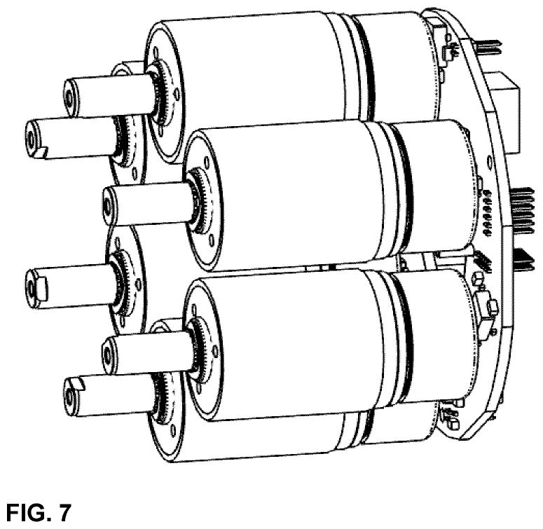

Referring to the drawings and more particularly to FIG. 1, an instrument drive mechanism for robotics in accordance with the present disclosure is generally shown at 10, and is occasionally referred to herein as mechanism 10 for simplicity. The instrument drive mechanism 10 is devised for supporting and driving an instrument. The mechanism 10 may interface the instrument to a robot arm, such as that shown at 11 in FIG. 8. In such an arrangement, the instrument may be known as an end effector. However, for simplicity, reference is made herein to an instrument, but this encompasses tools and/or end effectors of all types, such as gripping mechanism or gripper, anamorphic hand, endoscope, catheter, and tooling heads such as drills, saws, etc.

The robot arm 11 shown in FIG. 8 is an example of a possible type of robot supporting the instrument. Many other types of robotized systems can support the instrument with the mechanism 10, and therefore the mechanism 10 is not limited to the type of arm shown in FIG. 8. The robot arm 11 is a serial articulated robot arm, having an effector end 11A and a base end 11B. The effector end 11A is configured to receive thereon the mechanism 10 and any appropriate instrument. The base end 11B is configured to be connected to any appropriate structure or mechanism. The base end 11B may be rotatably mounted or not to the structure or mechanism. By way of non-exhaustive example, the base end 11B may be mounted to a wheelchair, to a vehicle, to a frame, to a cart, to a robot docking station. Although a serial robot arm is shown the joint arrangement of the robot arm 10 may be found in other types of robots, included parallel manipulators.

The robot arm 11 may have a series of links 12 (a.k.a., shells), interconnected by motorized joint units 13 (one shown in FIG. 8), with protective sleeves 14 at the junction between adjacent links 12:
The links 12 define the majority of the outer surface of the robot arm 11. The links 12 also have a structural function in that they form the skeleton of the robot arm 11 (i.e., an outer shell skeleton), by supporting the motorized joint units 13 and tools at the effector end 11A, with loads supported by the tools, in addition to supporting the weight of the robot arm 11 itself. Wires and electronic components may be concealed into the links 12, by internal routing. The open ends of the links 12 may each have a connector 12A for interconnection of links 12 with the motorized joint units 13, and with the mechanism 10.
The motorized joint units 13 interconnect adjacent links 12, in such a way that a rotational degree of actuation is provided between adjacent links 12. According to an embodiment, the motorized joint unit 13 shown in FIG. 8 is connected to the mechanism 10. The motorized joint units 13 may also form part of the structure of the robot arm 11, as they interconnect adjacent links 20.
The protective sleeves 14 shield the junction between pairs of adjacent links 12, e.g., in a water, fluid and particle resistant manner. The protective sleeves 14 may form a continuous fastener-less surface from one link 12 to another, as explained hereinafter. Although not shown to avoid interference, another protective sleeve 14 may be between at the junction of the mechanism 10 with the effector end 11A of the robot arm 11.

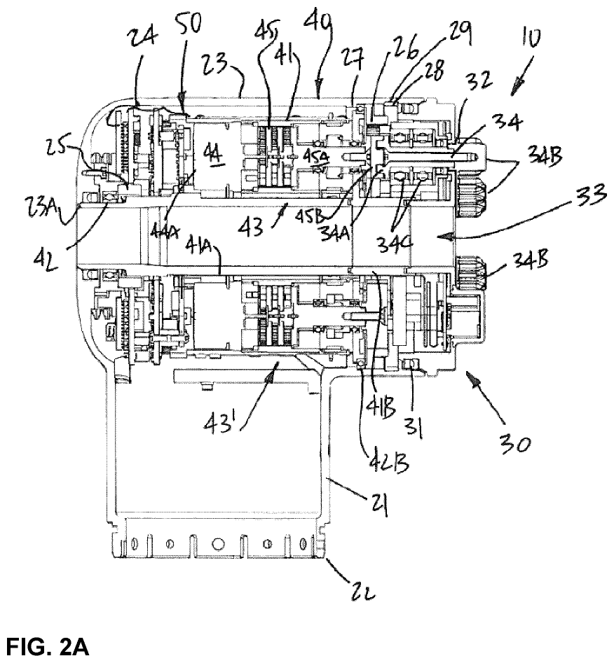

Referring now to FIGS. 1 and 2, the mechanism 10 has an outer shell 20, a interface cover 30, a drive system 40, and control boards 50. These components are divided in sub-components but generally form four main groups of the mechanism 10.
The outer shell 20 serves as a structural component of the mechanism 10, by which it is connected to the robot arm 11. Moreover, the outer shell 20 supports the various components inside the mechanism 10 including the interface cover 30, the drive system 40, and the control boards 50. The outer shell 20 will also support the weight of the instrument driven by the mechanism 10.
The interface cover 30 is the interface between the instrument and the outer shell 20 and drive system 40. The interface cover 30 therefore outputs the various degrees of actuation (DOAs) as explained hereinafter as received from the drive system 40. The interface cover 30 is also configured to rotate relative to the outer shell 20 to provide one rotational degree DOA to the instrument connected to the interface cover 30. The interface cover 30 is connected to the outer shell 20 so as to define a rotational joint. This DOA may be referred to as the roll of the mechanism 10.
The drive system 40 is tasked with driving the instrument connected to the mechanism 10 with the various DOAs provided by the mechanism 10, for instance in accordance with a robotic application or commands, or through user commands.
The control boards 50 (or control board unit of one or more printed circuit boards) support some of the electronic components tasked with operating the drive system 40. Moreover, the control boards 50 support sensors used to determine angular positions of the various rotational outputs of the drive system 40, and to determine the temperature of components of the drive system 40.

Referring to FIGS. 1 and 2, the outer shell 20, also known as skin, is shown as having an elbow-shaped tubular body 21, as one possible shape (e.g., tee shape, straight tube, L-shape, etc). At one end, the tubular body 21 has a connector 22 that is similar to the connector 12A of the exposed link 12 of the robot arm 11 in FIG. 8. The connectors 12A and 22 may be as described in U.S. Patent Application No. 62/479,841, incorporated herein by reference. Other connection arrangements are contemplated, with the connectors 12A and 22 given merely as an example. Therefore, the outer shell 20 may be connected to the robot arm 11 by the complementary connection with the motorized joint unit 13 and as covered by the protective sleeve 14, such that an orientation of the mechanism 10 relative to the robot arm 11 may be controlled by the motorized joint unit 13. This is one among numerous ways by which the outer shell 20 may be connected to a structure. As discussed previously, the mechanism 10 is not necessarily mounted to a robot arm 11. For example, a flange may be provided at the end of the outer shell 20 for connection to a structure or mechanism.

The outer shell 20 may further include an open-ended receptacle 23. The open-ended receptacle 23 has an open proximal end, while the distal end is generally closed, although the distal end may have a central bore 23A. The central bore 23A may be used to access an end of an instrument connected to the mechanism 10, among other possible uses. Other than for the central bore 23A which may or may not be represent, the outer surface of the outer shell 20 is generally smooth and without disruptions, such as fasteners holes. Also, an interior of the open-ended receptacle 23, i.e., its inner cavity, may open into an interior of the tubular body 21. This forms a continuous passage, notably for internal routing of cables.

Referring to FIGS. 2A and 2B, a slip ring 24 is located in the inner cavity of the open-ended receptacle 23, adjacent to the distal end of the receptacle 23. The slip ring 24 may include an annular body consisting of a plurality of contact rings (e.g., with grooves) for rotary contact with circuit components that are part of the control boards 50. For example, the slip ring 24 may have a printed circuit board upon which components are welded. The slip ring 24 may interface the various components of the mechanism 10 to a processing unit. In an embodiment, the slip ring 24 has electronic components so as to be autonomous and so as to be used to drive the mechanism 10 and the instrument it supports. Accordingly, as part of the slip ring 24, telecommunication hardware may be present for the mechanism 10 to receive control instructions. Adjacent to the slip ring 24, a magnetic ring 25 is present and will surround a shaft of the drive system 40. The magnetic ring 25 is fixed to the receptacle 23 via a bracket 25A, the magnetic ring 25 being used to determine the angular position of a shaft of the drive system 40 by way of a magnetic sensor on the control boards 50. The bracket 25A may be fixed to the receptacle 23 by fasteners 25B (one shown in FIG. 2B), or by any other attachment. The bracket 25A could also be an integral monolithic part of the receptacle 23 as well. For example, the magnetic sensor used with the magnetic ring 25 may be an absolute magnetic sensor that determines an angular position of the interface cover 30 by rotating with the interface cover 30. An absolute magnetic chip may be integrated to the magnetic ring 25. Other sensor types may be used, such as a rotary encoder.

An internal gear 26 is positioned in the inner cavity of the receptacle 23, toward the proximal open end. The internal gear 26 has its teeth oriented radially inward. The internal gear 26 may be connected to the open-ended receptacle 23 in any appropriate way. In an embodiment, as the receptacle 23 is without fasteners holes, the internal gear 26 is wedged immovably into the receptacle 23. For example, the internal gear 26 may be abutted against a rim 27 defined in an inner surface of the open-ended receptacle 23 and by a lock ring 28 received in a groove 29 also defined in the inner surface of the open-ended receptacle 23, and spaced apart from the rim 27 for the internal gear 26 to be fixed by the wedging, in such a way that the internal gear 26 is immovable relative to the receptacle 23. The absence of fasteners through the wall of the open-ended receptacle 23 may reduce wall thickness requirements. However, fasteners may also be present instead of the wedge arrangement that is described. Other fastening arrangements include welding, brazing, etc.

Referring to FIGS. 1, 2A, 2B and 4, the interface cover 30 is shown in detail. The interface cover 30 is mounted to the open-ended receptacle 23 so as to be rotatable relative to the open-ended receptacle 23. As seen in FIGS. 2A and 2B, the interface cover 30 has a bearing 31 that rotatingly supports it in the open-ended receptacle 23 of the outer shell 20. A rotational degree of freedom (DOF) is defined between the interface cover 30 and the open-ended receptacle 23.

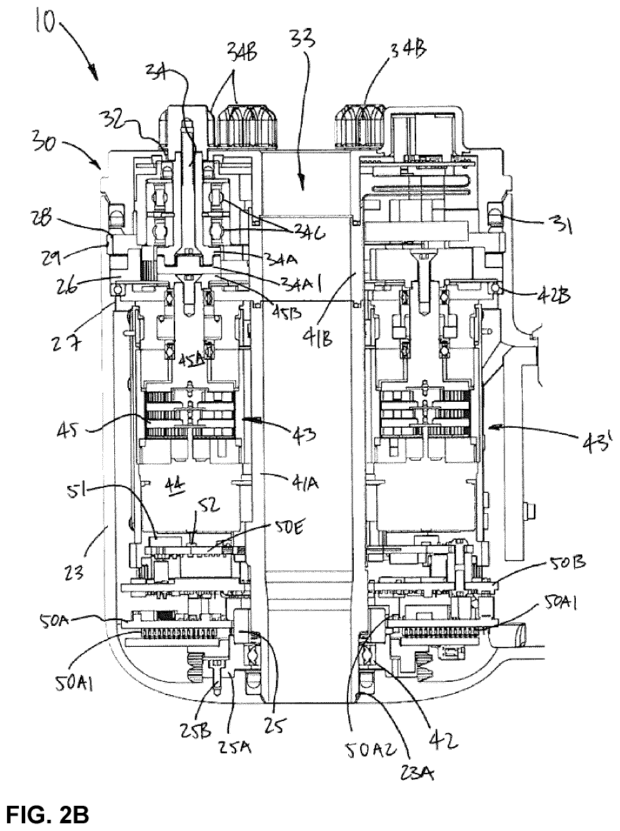

The interface cover 30 conceals the various components of the drive system 40 inside the open-ended receptacle 23. The interface cover 30 also serves a structural function in that it will interface the instrument to the drive system 40, and thus support the instrument. As is seen in FIG. 1, the interface cover 30 has a number of circumferentially-distributed bores 32, or circumferential bores 32, and a central bore 33. Shafts 34, which could each include a reduction gear box, are connected to and supported by the interface cover 30 and are aligned with the circumferential bores 32 such that their shafts project out of the circumferential bores 32. Couplers 34A may be provided at the distal end of the shafts 34, whereas output gears 34B are at the proximal end of the shafts 34. The shafts 34 could be without couplers 34A and extend straight into the motor units from the output gears 34B. The couplers 34A are used to connect the shafts 34 to motor units of the drive system 40, as described hereinafter. The couplers 34A may have any given shape. For example, the couplers 34A may be part of an Oldham coupling as shown in FIG. 3, with the coupler 34A defining a tongue projecting axially from a disc portion, for engagement with a groove in another disc 34A1, which disc 34A1 has grooves on its opposite faces, the grooves being perpendicular to one another. Another shape is a crown-shape, with a plurality of crenellations projecting from an annular base. The annular base is fixed to shaft 34, and the crenellations project axially for being coupled with a coupler having a corresponding shape. The output gears 34B are coupled to female components in the instrument so that the shafts 34 may transmit rotational DOAs to the instrument. The shafts 34 are each supported by one or more bearings 34C, to be stably supported by the interface cover 30. The bearings 34C are in relatively close proximity to the output gears 34B, minimizing the effect of any shaft deflection. As the instrument must be driven with precision, the presence of the shafts 34 in the interface cover 30, while an option, ensure a robust connection between the output gears 34B and female connectors in the instrument, for precise DOA transmission.

The central bore 33 is centered about a rotational axis of the interface cover 30. The central bore 33 is concentric with the central bore 23A, although respectively at the proximal end and the distal end of the receptacle 23. A boss 35 is also present and aligned in the circumference featuring the circumferential bores 31. The boss 35 may enclose an antenna for wireless communication between the mechanism 10 and the instrument it supports, for instance using radio frequency. This may for example allow identifying the type of instrument being used with the mechanism 10.

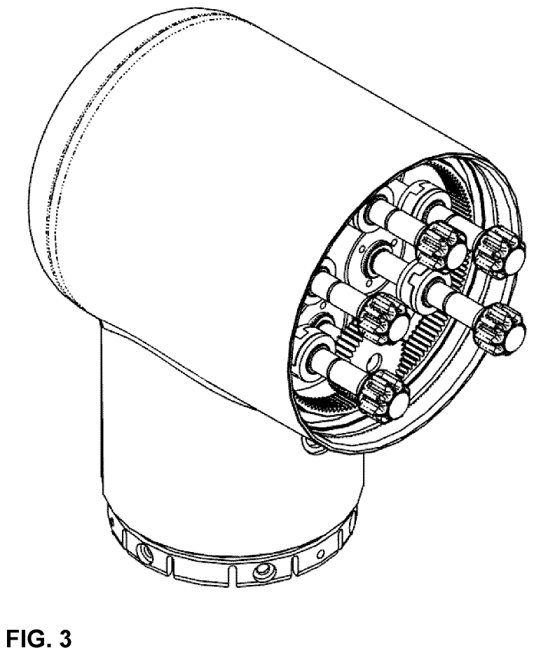

Various other connectors may be present on the cover 30 to assist in securing the instrument to the mechanism 10. For example, connectors such as fastener bores 36A and alignment slot 36B may be defined in the cover. Referring to FIG. 4, the interface cover 30, including its various components may be in the form a cartridge, thereby facilitating its insertion and removal from the open-ended receptacle 23. Such a configuration may facilitate the maintenance and repair of the mechanism 10.

The drive system 40 provides the various DOAs of the mechanism 10 to the instrument. In the illustrated embodiment, and more particularly in FIG. 1, five different output gears 34B are shown, each one of which is associated with an independent DOA. Moreover, the interface cover 30 has been described above as being drivable in roll, whereby a sixth DOA is provided. Fewer or more DOAs may be provided by the mechanism 10. In an embodiment, in addition to the roll, a pair of the output gears 34B is used for a pitch control of the instrument, another pair of the output gears 34B may be used for yaw control, and a last of the output gears 34B may be used for any other motion of the instrument. Other distributions of the DOAs are of course possible, depending on the contemplated use and nature of the instrument.

The drive system 40 has a frame 41 that structurally supports the various components of the drive system 40. The frame 41 is rotatably mounted into the open-ended receptacle 23 of the outer shell 20, and is connected to the interface cover 30 so as to rotate with it. As shown in FIGS. 2A and 2B, the frame 41 may have various components. For example, the frame 41 may include a central shaft 41A supporting a bearing 42 that will be received at the distal end of the open-ended receptacle 23. The central shaft 41A may form a continuous passage with the central bore 33 of the interface cover 30, via an inner cavity of the central shaft 41A. The frame 41 may further include a hub and flange 41B connected axially to the central shaft 41A, and featuring another bearing 42B to rotatingly support the frame 41 nearer to the proximal end of the open-ended receptacle 23.

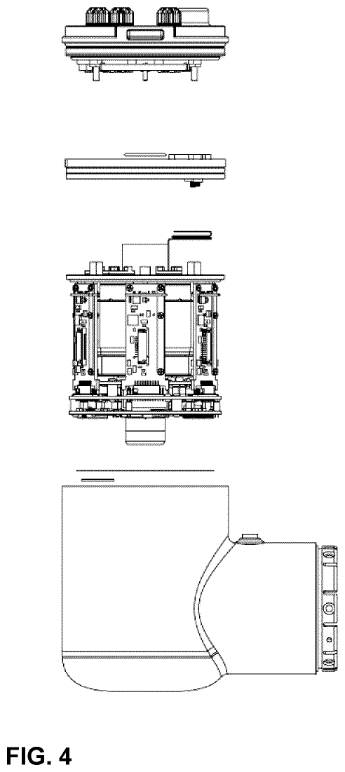

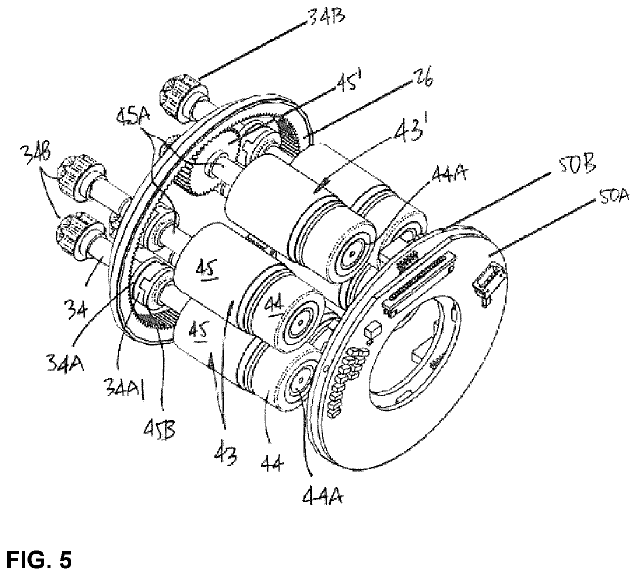

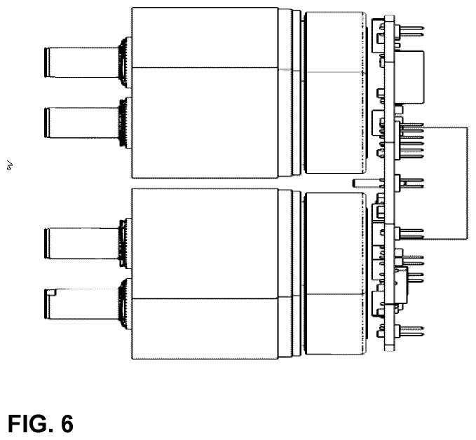

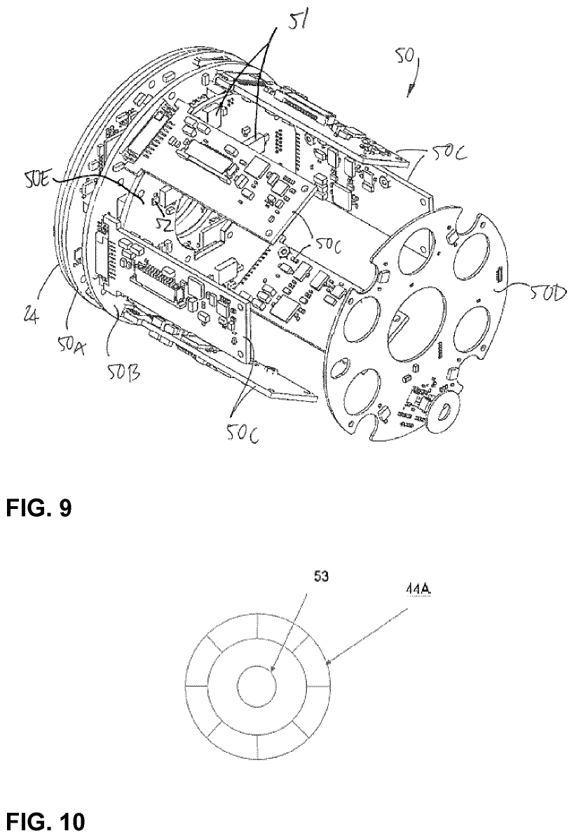

As best seen in FIGS. 5-7, the drive system 40 may comprise a plurality of motor units 43. In the illustrated embodiment, the drive system 40 has five motor units 43 and one illustrated as 43′. The motor units 43 and 43′ will provide the various DOAs of the drive system 40. According to an embodiment, the motor units 43 and 43′ include a motor 44 having a sensor ring 44A (which could also be a sensor wheel) at its distal end. The sensor ring 44A has lines thereon that may be read by an optical sensor described herein, to determine an angular position (a.k.a., an orientation) of the rotor end of the motor unit 43, with FIG. 10 showing an example of such lines, though fewer or more lines could be present. The motors 44 may be electric motors, for instance uni-directional or bi-directional rotation capacity. A shaft (not shown) of the motor 44 is coupled to a gear box 45 (a.k.a., gear head) having a shaft 45A. A coupler 45B is at a proximal end of the motor units 43 for complementary coupling with the couplers 34A and/or the disc 34A1 of the shaft 34 in the interface cover 30, for transmission of rotation and torque from one to another. The complementary coupling may be self-adjusting, for instance by wedging engagement, to assist in the coupling when the interface cover 30 is inserted as a cartridge in the open-ended receptacle 23. According to an embodiment, the couplers 34A and 45B are the same (both having the same tongue size), and form part of an Oldham coupling with the disc 34A1.

The motor unit 43′ is not coupled to one of the shafts 34. The motor unit 43′ has a gear 45′ mounted to its proximal end. The gear 45′ is meshed with the internal gear 26 fixed to the outer shell 20. It is therefore the motor unit 43′ that drives a rotation of the interface cover 30 and the drive system 40 relative to the outer shell 20. The gear 45′ may have an extension journaled into the interface cover 30 for the opposite sides of the gear 45′ to be rotatingly supported.

In similar fashion to the interface cover 30, the drive system 40, as shown in FIG. 4, may be provided as a cartridge fitted into the open-ended receptacle 23 from the proximal open end, also to facilitate its insertion and removal from the open-ended receptacle 23. Such a configuration may facilitate the maintenance and repair of the drive system 40 of the mechanism 10. It may also help to connect simultaneously the slip ring 24, bearing and distal seal in a single motion of insertion.

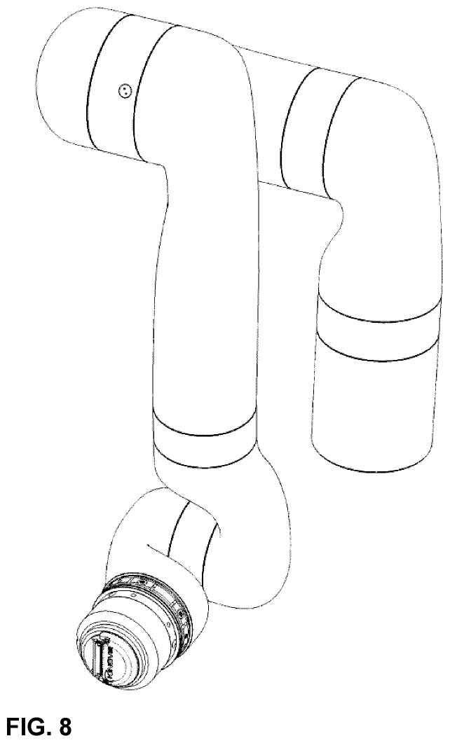

Referring to FIGS. 4-8, the control boards 50 are shown as including various printed circuit boards (PCBs) supporting electronics component to drive the drive system 40, and the instrument, in collaboration with the slip ring 24. As seen in FIG. 5, the control boards 50 may include a master board 50A, adjacent to and in contact with the slip ring 24, via brushes 50A1 or like contacts. Optionally, a slave board 50B is connected to the master board 50A and supports the various sensors as described hereinafter, as well as driver boards 50C (FIG. 9) for each of the motor units 43 and 43′. One sensor that may be supported by the master board 50 is magnetic sensor 50A2. The magnetic sensor 50A2 may be an absolute magnetic sensor. As it is on the master board 50, it rotates with it, and thus it rotates concurrently with the interface cover 30. Its cooperation with the magnetic ring 25 allows a determination of the angular position of the interface cover 30. In an embodiment, the magnetic ring 25 and the magnetic sensor 50A2 are in a same axial plane of the shaft 41A of the drive system 40. In an embodiment, the magnetic sensor 50A2 is radially outward of the magnetic ring 25. In a space-efficient arrangement, the driver boards 50C are transversely mounted onto the slave board 50B (or to the master board 50A in the absence of a slave board 50B). An RFID board 50D may be adjacent to the interface cover 30 to operate the RFID antenna. According to an embodiment, another sensor board 50E may be present, of smaller diametrical dimensions than the boards 50A and 50B (if present), to be in closer proximity to the motor units 43/43′. For example, the sensor board 50E may be lodged between the driver boards 50C. In an embodiment, the planes of the driver boards 50C are perpendicular both the planes of the master board 50A, of the slave board 50B, and of the sensor board 50E. The planes of the master board 50A, of the slave board 50B and/or of the sensor board 50E may be parallel to one another.

The control boards 50 are located between the slip ring 24 and the drive system 40, with the driver boards 50C projecting alongside the motor units 43 and 43′ in the drive system 40. The control boards 50 are coupled to the drive system 40, to rotate with it, and may be part of the cartridge assembly of the drive system 40 that may come assembled with the drive system 40 for installation into the outer shell 20. Therefore, the slip ring 24 establishes contact between the control boards 50 and the robotic arm 11 to power the control boards 50 who then power the drive system 40.

Moreover, the control boards 50 supports sensors, in an effort to minimize space taken by sensors. For example, the sensors may be on the sensor board 50E to be in close proximity to the motor units 43/43′. Optical encoders 51, also known as position sensors, and temperature sensors 52 are integrated into the sensor board 50E, if present, or integrated in the master board 50A or slave board 50B in the absence of a sensor board 50E. Both sensor types are contactless and fit into the limited space between the sensor board 50E and the drive system 40. The optical encoders 51 are each paired with an opposite the sensor wheels 44A, to track an angular position of the outputs at the output gears 34.

The optical encoders 51 in an embodiment are a reflective-based optical encoders. For accuracy, the sensor rings 44A paired with the optical encoders 51 are code wheels or rings, and may be glued to a rotor or shaft of the motor units 43 and 43′. In an embodiment, a custom tool is used for the gluing to assure pre-determined positioning is achieved and to minimize the non-linearity in reading the position.

The temperature sensors 52 may for instance use infrared thermopile technology. The infrared temperature sensors 52 require a non-reflective surface for optimal results. Since the sensor rings 44A and the rotor of the motor units 43 and 43′ are made of reflective materials, a pad 53 of a dark and mat colour may be present. In an embodiment, as shown in FIG. 10, the pad 53 is a non-reflective adhesive-backed polymer added to the motor units 43 and 43′, and designed to fit inside the sensor rings 44A′s inner diameter to reduce reflection where the temperature sensor 52 is aimed. Accordingly, the infrared sensors 52 are aligned with the axis of the motor units 43 and 43′ while the optical encoders 51 are off-axis. The centered position of the temperature sensors 52 is advantageous from a temperature point of view due to the fact that the temperature on the rotor of the motor units 43 and 43′ is read, and the heat path is through the shaft of the motor units 43 and 43′. The infrared sensors 52 may also be off-axis in an embodiment. In accordance with an embodiment, each of the temperature sensors 52 monitor individually the temperature of the motor units 43/43′. When one of the temperature sensors 52 detects a temperature above a determined level, a fault signal is generated. Consequently, the control boards 50 may communicate the fault signal to the drive system 40. As a response, the drive system 40 may brake movement of all various motorized joints of the robot arm 11 and the mechanism 10 may pause its operation. This is one possibility among others. The temperature threshold may be set so as to avoid overheating, and excessive temperatures that could impact the function of the various chips on the control boards 50, and could affect the magnets and the windings of the motor units 43 and 43′.

For the optical encoders 51, they have to be placed within distance tolerances of the sensor rings 44A. A riser PCB may thus support the optical encoders 51, to then be installed on the larger PCB of the control boards 50 in order to raise the optical encoder chip close to the motor units 43 and 43′, above all other chips. In the illustrated embodiment, the control boards 50 have a total of 12 sensors (six optical encoders 51 and six temperature sensors 52) in a small and uncluttered package, for the five motor units 43 and one motor unit 43′. Each of the motor units 43 and 43′ may have a dedicated set of sensor ring 44A and optical encoder 51, and/or a dedicated temperature sensor 52, and/or a dedicated set of temperature sensor 52 and pad 53. All of these components may be located in a space defined between a supporting board from among the control boards 50, such as the master board 50A, slave board 50B or sensor board 50E, and the motor units 43 and 43′. In an embodiment, all of these components may be located in a space defined by a plane of the master board 50A, of the slave board 50B or of the sensor board 50E, and a plane including ends of the motor units 43 and 43′. The arrangement of the sensors 51 and 52 on the sensor board 50E simplifies the assembly.

1. An instrument drive mechanism comprising:
an outer shell having an open-ended receptacle;
an internal gear secured inside the open-ended receptacle and immovable relative to the outer shell;
an interface cover rotatably mounted to the open-ended receptacle, the interface cover configured to be connected to an instrument, the interface cover rotatably supporting at least one cover shaft with an output adapted to be rotatingly coupled to the instrument;
a drive system rotatably mounted to the open-ended receptacle and connected to the interface cover to rotate with the interface cover, the drive system having
at least two motor units,
a coupling assembly between each of the at least one cover shaft and a corresponding one of the motor units for releasably coupling a motor unit shaft to the cover shaft, for the at least one said motor unit coupled to each said at least one cover shaft to transmit a degree of actuation thereto, and one said motor unit having a gear coupled to internal gear to drive a rotation of the interface cover and drive system relative to the outer shell.

2. The instrument drive mechanism according to claim 1, wherein the coupling assembly includes at least a coupler connected to the cover shaft, and a coupler receiving a drive from the motor unit.

3. The instrument drive mechanism according to claim 1, wherein each of said coupling assembly is an Oldham coupling.

4. The instrument drive mechanism according to claim 1, wherein each motor unit has a motor and a reduction gear box (RGB) connected to the motor, a RGB shaft being coupled to the cover shaft by the coupling assembly.

5. The instrument drive mechanism according to claim 1, wherein each said cover shaft is connected to the interface cover by at least one bearing.

6. The instrument drive mechanism according to claim 1, comprising at least two of the cover shaft each with one said output, with one said coupling assembly between each of the two cover shafts and a corresponding one of the motor units.

7. The instrument drive mechanism according to claim 1, wherein the interface cover, the at least one shaft and a coupler of coupling assembly form a cartridge removable as a group from the outer shell and from engagement with the drive system.

8. The instrument drive mechanism according to claim 1, further comprising at least one bearing between an inner surface of the outer shell and a periphery of the interface cover.

9. The instrument drive mechanism according to claim 1, further comprising a central shaft extending into the outer shell and rotatably supported by the outer shell, the interface cover and the drive system coupled to the central shaft to rotate concurrently with the central shaft.

10. The instrument drive mechanism according to claim 9, further comprising a sensor unit having a sensor portion mounted onto a printed circuit board (PCB) connected to the drive system and/or to the shaft to monitor a rotation of the shaft and/or of the drive system relative to the outer shell to determine an angular position of the interface cover relative to the outer shell.

11. The instrument drive mechanism according to claim 10, wherein the sensor portion of the PCB is a magnetic sensor.

12. The instrument drive mechanism according to claim 11, further comprising a magnetic ring secured to the outer shell and surrounding the central shaft adjacent to the magnetic sensor.

13. The instrument drive mechanism according to claim 12, wherein the magnetic sensor and the magnetic ring lie in a common radial plane of the central shaft.

14. The instrument drive mechanism according to claim 12, wherein the magnetic sensor is radially outward of the magnetic sensor.

15. The instrument drive mechanism according to claim 10, wherein the interface cover has a central bore, the central bore of the interface cover forming a continuous passage with an inner cavity of the central shaft.

16. The instrument drive mechanism according to claim 1, further comprising at least one printed circuit board connected to the drive system, the printed circuit board supporting a temperature sensor for each said motor unit, and an optical encoder for each said motor unit to determine an angular position of each said output on the interface cover.

17. The instrument drive mechanism according to claim 16, wherein the temperature sensor is an infrared temperature sensor.

18. The instrument drive mechanism according to claim 17, wherein the infrared temperature sensor is aligned with a shaft of its corresponding motor unit.

19. The instrument drive mechanism according to claim 16, further comprising a pad rotating with a shaft of the motor unit, the pad paired with the optical encoder.

20. The instrument drive mechanism according to claim 16, wherein the optical encoder is located offset relative to a center of a shaft of the motor unit.

21.-31. (canceled)
